(19) United States
(12) Patent Application Publication
(10) Pub. No.: US 2026/0179769 A1
(43) Pub. Date: Jun. 25, 2026

(54) GENERATING AND INTEGRATING COVARIATES INTO MODELS

(71) Applicant: International Business Machines Corporation, Armonk, NY (US)

(72) Inventors: Uri Kartoun, Cambridge, MA (US); Joao H. Bettencourt-Silva, Dublin (IE); Natalia Mulligan, Dublin (IE); Vibha Anand, Chestnut Hill, MA (US)

(21) Appl. No.: 19/000,332

(22) Filed: Dec. 23, 2024

(51) Int. Cl. G16H 50/20; G16H 20/10; G16H 50/70

(52) U.S. Cl. CPC G16H 50/20; G16H 20/10; G16H 50/70

(57) ABSTRACT

A method, according to one approach, includes: defining ingredients and brand names of the ingredients in a set of drugs, and calculating lengths of use of the respective drugs in the set. The method also includes classifying types of the drugs, and the respective lengths of use, to a corresponding duration class. New covariates are constructed based at least in part on the types of drugs and/or the respective lengths of use. The new covariates and standard covariates are incorporated into a prediction model, and any effect(s) the new covariates have on an outcome of the prediction model are assessed.

### STATEMENT REGARDING PRIOR DISCLOSURES BY THE INVENTOR OR A JOINT INVENTOR

The following disclosure(s) are submitted under 35 U.S.C. 102(b)(1)(A):
DISCLOSURE(S): Kartoun, U., Koseki, A., Kosugi, A. et al. Investigating the impact of steroid dependence on gastrointestinal surgical outcomes from UK Biobank. Sci Rep 14, 29243 (2024). Published Nov. 25, 2024. https://doi.org/10.1038/s41598-024-75215-5.

### BACKGROUND

The present invention relates to artificial intelligence (AI) based models, and more specifically, this invention relates to developing covariates for AI based models.

AI based models have emerged in recent years, allowing the development of highly accurate clinical risk assessment tools. While different types of AI based models are able to evaluate inputs differently depending on the situation, the insight the inputs provide depends on how extensive the inputs are. For instance, an availability and quality of covariates may impact the level of detail that may be achieved by an AI based model evaluating input datapoints and/or making a decision. With respect to the present description, a “covariate” includes any variable(s) in addition to the variable(s) of interest that may be considered while analyzing a set of data.

### SUMMARY

A method, according to one approach, includes: defining ingredients and brand names of the ingredients in a set of drugs, and calculating lengths of use of the respective drugs in the set. The method also includes classifying types of the drugs, and the respective lengths of use, to a corresponding duration class. New covariates are constructed based at least in part on the types of drugs and/or the respective lengths of use. The new covariates and standard covariates are incorporated into a prediction model, and any effect(s) the new covariates have on an outcome of the prediction model are assessed.

A computer program product, according to another approach, includes: one or more computer-readable storage media. The computer program product also includes program instructions that are stored on the one or more storage media to perform the foregoing method.

A computer system, according to yet another approach, includes: a processor set, and one or more computer-readable storage media. The computer system also includes program instructions that are stored on the one or more storage media to cause the processor set to perform the foregoing method.

Other aspects and implementations of the present invention will become apparent from the following detailed description, which, when taken in conjunction with the drawings, illustrate by way of example the principles of the invention.

### BRIEF DESCRIPTION OF THE DRAWINGS

FIG. 1 is a diagram of a computing environment, in accordance with one approach.

FIG. 2 is a representational view of a distributed system, in accordance with one approach.

FIG. 3A is a flowchart of a method, in accordance with one approach.

FIG. 3B is a flowchart of a method, in accordance with one approach.

FIG. 4A is a table illustrating how details corresponding to a patient may be evaluated to determine the dosage and/or quantity of a drug, in accordance with an in-use example.

FIG. 4B is a graph illustrating exemplary duration classes for use of a drug, in accordance with an in-use example.

FIG. 4C is a graph illustrating the impact that taking different prescription drugs for different lengths of time will have on a patient, in accordance with an in-use example.

### DETAILED DESCRIPTION

The following description is made for the purpose of illustrating the general principles of the present invention and is not meant to limit the inventive concepts claimed herein. Further, particular features described herein can be used in combination with other described features in each of the various possible combinations and permutations.

Unless otherwise specifically defined herein, all terms are to be given their broadest possible interpretation including meanings implied from the specification as well as meanings understood by those skilled in the art and/or as defined in dictionaries, treatises, etc.

It must also be noted that, as used in the specification and the appended claims, the singular forms “a,” “an” and “the” include plural referents unless otherwise specified. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The following description discloses several preferred approaches of systems, methods and computer program products for creating new covariates and integrating them into AI based models to provide more detailed insight. These new covariates gather new insights regarding the impact they have on determinations that are made by the one or more models. For example, covariates corresponding to the length of time a drug (e.g., prescription drug) have been taken by a patient with medical issue(s) may be integrated into one or more AI based models that are able to make predictions on whether actions taken now will increase or decrease the chances of a major surgery in the future to treat the medical issue(s). Approaches herein may thereby be modified to improve the process of prescribing medical treatment (e.g., medical prescriptions) and/or determining whether to cause the medical treatment to be administered to a patient, e.g., as will be described in further detail below.

In one general approach, a method includes: defining ingredients and brand names of the ingredients in a set of drugs, and calculating lengths of use of the respective drugs in the set. The method also includes classifying types of the drugs, and the respective lengths of use, to a corresponding duration class. New covariates are constructed based at least in part on the types of drugs and/or the respective lengths of use. The new covariates and standard covariates are incorporated into a prediction model, and any effect(s) the new covariates have on an outcome of the prediction model are assessed.

In another general approach, a computer program product includes: one or more computer-readable storage media. The computer program product also includes program instructions that are stored on the one or more storage media to perform the foregoing method.

In yet another general approach, a computer system includes: a processor set, and one or more computer-readable storage media. The computer system also includes program instructions that are stored on the one or more storage media to cause the processor set to perform the foregoing method.

Various aspects of the present disclosure are described by narrative text, flowcharts, block diagrams of computer systems and/or block diagrams of the machine logic included in computer program product (CPP) approaches. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product approach (“CPP approach” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as improved covariate code at block 150 for creating new covariates and integrating them into AI based models to provide more detailed insight. These new covariates gather new insights regarding the impact they have on determinations that are made by the one or more models. For example, covariates corresponding to the length of time a drug (e.g., prescription drug) has been taken by a patient with medical issue(s) may be integrated into one or more AI based models that are able to make predictions on whether actions taken now will increase or decrease the chances of a major surgery in the future to treat the medical issue(s). Approaches herein may thereby be modified to improve the process of prescribing medical treatment (e.g., medical prescriptions) and/or determining whether to cause the medical treatment to be administered to a patient, e.g., as will be described in further detail below.

In addition to block 150, computing environment 100 includes, for example, computer 101, wide area network (WAN) 102, end user device (EUD) 103, remote server 104, public cloud 105, and private cloud 106. In this approach, computer 101 includes processor set 110 (including processing circuitry 120 and cache 121), communication fabric 111, volatile memory 112, persistent storage 113 (including operating system 122 and block 150, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144. In some approaches, the EUD 103 is a medical device configured to collect data (e.g., readings, samples, etc.) from patients and/or to be used by a medical professional (e.g., doctor, nurse, etc.) to enter patient data. For instance, data specific to a patient may be converted into one or more covariates, and integrated into one or more AI based models such that the models are trained to identify patterns, make predictions, etc., regarding a patient's medical condition(s). According to one example, the EUD 103 may be used to collect and submit details, e.g., such as how long a prescription drug has been (or is planned to be) taken by a patient in order to treat an underlying medical condition, which are used to train AI based models to be able to predict whether a prescription should be extended for a patient. For instance, the AI based models may be able to determine whether continued usage of the prescription drug will have a material impact on whether surgery is prescribed in the future to treat the underlying medical condition, e.g., as will be described in further detail below.

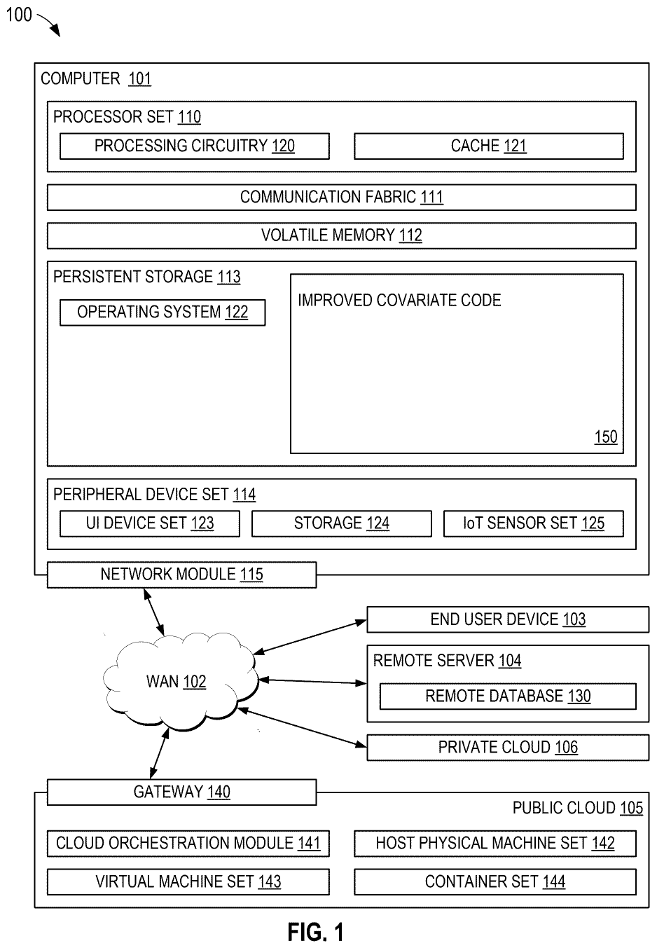

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 150 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 150 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various approaches, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some approaches, storage 124 may take the form of a quantum computing storage device for storing data in the form of qubits. In approaches where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer, and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some approaches, network control functions and network forwarding functions of network module 115 are performed on the same physical hardware device. In other approaches (for example, approaches that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some approaches, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some approaches, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other approaches a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this approach, public cloud 105 and private cloud 106 are both part of a larger hybrid cloud.

CLOUD COMPUTING SERVICES AND/OR MICROSERVICES (not separately shown in FIG. 1): private and public clouds 106 are programmed and configured to deliver cloud computing services and/or microservices (unless otherwise indicated, the word “microservices” shall be interpreted as inclusive of larger “services” regardless of size). Cloud services are infrastructure, platforms, or software that are typically hosted by third-party providers and made available to users through the internet. Cloud services facilitate the flow of user data from front-end clients (for example, user-side servers, tablets, desktops, laptops), through the internet, to the provider's systems, and back. In some approaches, cloud services may be configured and orchestrated according to as “as a service” technology paradigm where something is being presented to an internal or external customer in the form of a cloud computing service. As-a-Service offerings typically provide endpoints with which various customers interface. These endpoints are typically based on a set of APIs. One category of as-a-service offering is Platform as a Service (PaaS), where a service provider provisions, instantiates, runs, and manages a modular bundle of code that customers can use to instantiate a computing platform and one or more applications, without the complexity of building and maintaining the infrastructure typically associated with these things. Another category is Software as a Service (SaaS) where software is centrally hosted and allocated on a subscription basis. SaaS is also known as on-demand software, web-based software, or web-hosted software. Four technological sub-fields involved in cloud services are: deployment, integration, on demand, and virtual private networks.

In some aspects, a system according to various approaches may include a processor and logic integrated with and/or executable by the processor, the logic being configured to perform one or more of the process steps recited herein. The processor may be of any configuration as described herein, such as a discrete processor or a processing circuit that includes many components such as processing hardware, memory, I/O interfaces, etc. By integrated with, what is meant is that the processor has logic embedded therewith as hardware logic, such as an application specific integrated circuit (ASIC), a FPGA, etc. By executable by the processor, what is meant is that the logic is hardware logic; software logic such as firmware, part of an operating system, part of an application program; etc., or some combination of hardware and software logic that is accessible by the processor and configured to cause the processor to perform some functionality upon execution by the processor. Software logic may be stored on local and/or remote memory of any memory type, as known in the art. Any processor known in the art may be used, such as a software processor module and/or a hardware processor such as an ASIC, a FPGA, a central processing unit (CPU), an integrated circuit (IC), a graphics processing unit (GPU), etc.

Of course, this logic may be implemented as a method on any device and/or system or as a computer program product, according to various approaches.

As noted above, AI based models have emerged in recent years, allowing the development of highly accurate clinical risk assessment tools. While different types of AI based models are able to evaluate inputs differently depending on the situation, the insight the inputs provide depends on how extensive the inputs are. For instance, an availability of covariates may impact the level of detail that may be achieved by an AI based model evaluating input datapoints and/or making a decision. With respect to the present description, a “covariate” includes any variable(s) in addition to the variable(s) of interest that may be considered while analyzing a set of data. It should be noted that in some approaches, “covariate” may be used interchangeably with “features,” “attributes,” and “factors” of AI based models. According to a non-limiting example, when analyzing the dependency between a blood protein and a given diagnosis of a patient, an age of the patient may serve as a covariate. In a model trained to predict blood protein levels based on a diagnosis, the patient's age may be useful in determining whether variance unrelated to the diagnosis is at play. Thus, the age, blood pressure, etc. of a patient may serve as covariates, where the older the patient is and/or the higher the patient's blood pressure is, the more likely they will go through a major surgery.

Conventional models typically only consider covariates that are predetermined datapoints, e.g., such as lab values, comorbidities, age, ethnicity, etc. of a patient. Conventional models are thereby limited to making simple (and often inaccurate) predictions. In sharp contrast to shortcomings experienced by conventional models, approaches herein are desirably able to determine novel covariates and combinations thereof that are further incorporated into AI based models. These new covariates may be evaluated and utilized to improve performance of AI based models.

The integration of new and useful covariates into a prediction model improves the performance of the model. For example, existing novel time dependent covariates (e.g., such as stimulus-response) may be developed in approaches herein and used to gather new insights regarding the impact they have on determinations made by one or more AI based models. The ability to develop insight with these novel covariates have further been verified with experimentation, e.g., as will be described in further detail below.

It should be noted that although certain approaches herein are described in the context of medical conditions involving Gastrointestinal (GI) medical issues, this is in no way intended to be limiting. For example, approaches herein involve evaluating prescriptions for drugs that are configured to treat symptoms of inflammatory bowel diseases (IBDs). However, novel covariates may be developed between any desired datapoints pertaining to a given patient, providing insight to details of a specific medical issue experienced by the given patient. For example, covariates corresponding to the length of time a drug (e.g., prescription drug) was taken by a patient treating their medical issue(s) may be integrated into one or more AI based models that are able to make predictions on whether actions taken now will increase or decrease the chances of a major surgery in the future to treat the medical issue(s). Approaches herein may thereby be modified to improve the process of prescribing medical treatment (e.g., prescription drugs) and/or determining whether to cause the medical treatment to be administered to a patient, e.g., as will be described in further detail below. It should also be noted that use of the term “drug” herein is in no way intended to be limiting either. Approaches herein may be applied in situations involving medications, natural medicinal remedies, non-prescription drugs, prescription drugs, etc.

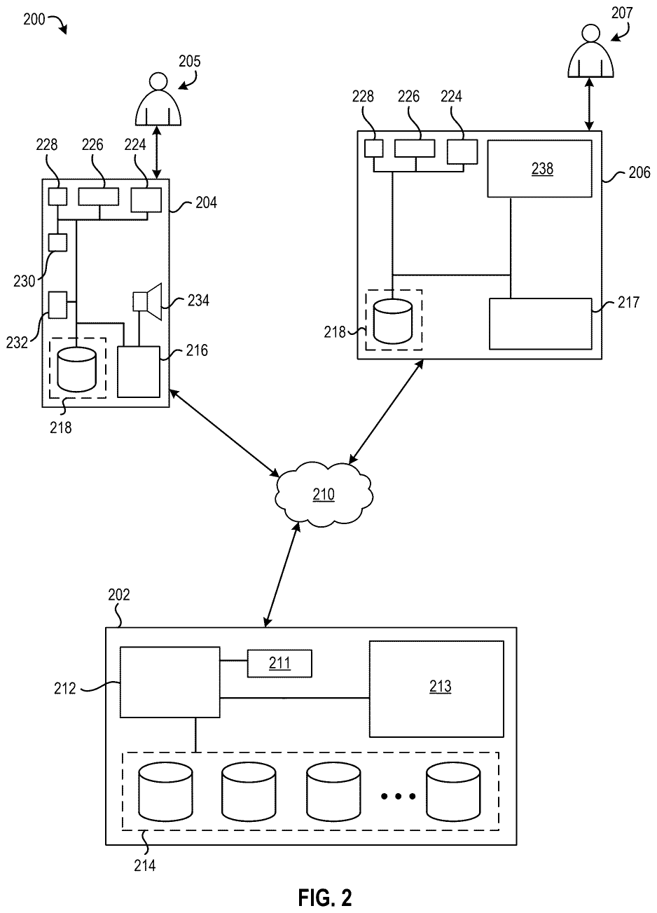

Looking now to FIG. 2, a system 200 having a distributed architecture is illustrated in accordance with one approach. As an option, the present system 200 may be implemented in conjunction with features from any other approach listed herein, such as those described with reference to the other FIGS., such as FIG. 1. However, such system 200 and others presented herein may be used in various applications and/or in permutations which may or may not be specifically described in the illustrative approaches or implementations listed herein. Further, the system 200 presented herein may be used in any desired environment. Thus FIG. 2 (and the other FIGS.) may be deemed to include any possible permutation.

As shown, the system 200 includes a central server 202 that is connected to a remote device 204, and edge node 206 accessible to the user 205 and administrator 207, respectively. The remote device 204 and edge node 206 may thereby be considered endpoint devices, each of which are connected to the central server 202. For example, the remote device 204 may be a laptop belonging to (e.g., accessible by) a medical professional. The laptop may include hardware and/or software that are able to communicate with programs running on the edge node 206 and/or central server 202. The remote device 204 may thereby be used by user 205 (e.g., a doctor) to update medical information pertaining to a patient being examined, e.g., as will be described in further detail below.

The central server 202, remote device 204, and edge node 206 are each connected to a network 210, and may thereby be positioned in different geographical locations. The network 210 may be of any type, e.g., depending on the desired approach. For instance, in some approaches the network 210 is a WAN, e.g., such as the Internet. However, an illustrative list of other network types which network 210 may implement includes, but is not limited to, a LAN, a PSTN, a SAN, an internal telephone network, etc. As a result, any desired information, data, commands, instructions, responses, requests, etc. may be sent between remote device 204, edge node 206, and/or central server 202, regardless of the amount of separation which exists therebetween, e.g., despite being positioned at different geographical locations. According to some approaches, the central server 202 is a remote cloud server that is connected to (e.g., may be accessed by) remote device 204 and/or edge node 206.

However, it should be noted that two or more of the remote device 204, edge node 206, and central server 202 may be connected differently depending on the approach. According to an example, which is in no way intended to limit the invention, two servers (e.g., nodes) may be located relatively close to each other and connected by a wired connection, e.g., a cable, a fiber-optic link, a wire, etc.; etc., or any other type of connection which would be apparent to one skilled in the art after reading the present description.

The terms “user” and “administrator” are in no way intended to be limiting either. For instance, while users and administrators may be described as being individuals (e.g., patients, doctors, medical professionals, etc.) in various implementations herein, a user and/or an administrator may be an application, an organization, a preset process, etc. The use of “data,” “metadata,” and “information” herein are in no way intended to be limiting either, and may include any desired type of details, e.g., depending on the type of operating system implemented on the remote device 204, edge node 206, and/or central server 202. In some approaches, readings that are taken by logical and/or physical components at the edge node 206 and/or remote device 204 may be kept at the edge node 206 for evaluation using one or more AI based models, e.g., as will soon become apparent.

The central server 202 includes a large (e.g., robust) processor 212 coupled to a cache 211, an AI module 213, and a data storage array 214 having a relatively high storage capacity. The AI module 213 may include any desired number and/or type of AI-based models, e.g., such as machine learning models, deep learning models, neural networks, etc. In preferred approaches, the AI module 213 includes one or more models that have been trained to develop and/or implement novel covariates. These new covariates gather new insights regarding the impact they have on determinations that are made by the one or more models. For example, covariates corresponding to the length of time a drug (e.g., prescription drug) has been taken by a patient with medical issue(s) may be integrated into one or more AI based models that are able to make predictions on whether actions taken now will increase or decrease the chances of a major surgery in the future to treat the medical issue(s). Approaches herein may thereby be modified to improve the process of prescribing medical treatment (e.g., medical prescriptions) and/or determining whether to cause the medical treatment to be administered to a patient, e.g., as will be described in further detail below.

With continued reference to FIG. 2, remote device 204 includes a processor 216 which is coupled to memory 218. The processor 216 receives inputs from and interfaces with user 205. For instance, the user 205 may input information and/or queries using one or more of: a display screen 224, keys of a computer keyboard 226, a computer mouse 228, a microphone 230, and a camera 232. The processor 216 may thereby be configured to receive inputs (e.g., text, sounds, images, motion data, etc.) from any of these components as entered by the user 205. These inputs typically correspond to information presented on the display screen 224 while the entries were received. Moreover, the inputs received from the keyboard 226 and computer mouse 228 may impact the information shown on display screen 224, data stored in memory 218, information collected from the microphone 230 and/or camera 232, status of an operating system being implemented by processor 216, etc. The electronic device 204 also includes a speaker 234 which may be used to play (e.g., project) audio signals for the user 205 to hear.

Looking now to the edge node 206, some of the components included therein may be the same or similar to those included in remote device 204, some of which have been given corresponding numbering. For instance, controller 217 is coupled to memory 218, a display screen 224, keys of a computer keyboard 226, and a computer mouse 228. Additionally, the controller 217 is coupled to an AI module 238. As described above with respect to AI module 213, the AI module 238 may include one or more historical question-answer modelers that are able to develop and/or implement novel covariates. These new covariates gather new insights regarding the impact they have on determinations that are made by the one or more models. For example, covariates corresponding to the length of time a drug (e.g., prescription drug) has been taken by a patient with medical issue(s) may be integrated into one or more AI based models that are able to make predictions on whether actions taken now will increase or decrease the chances of a major surgery in the future to treat the medical issue(s). Approaches herein may thereby be modified to improve the process of prescribing medical treatment (e.g., medical prescriptions) and/or determining whether to cause the medical treatment to be administered to a patient, e.g., as will be described in further detail below.

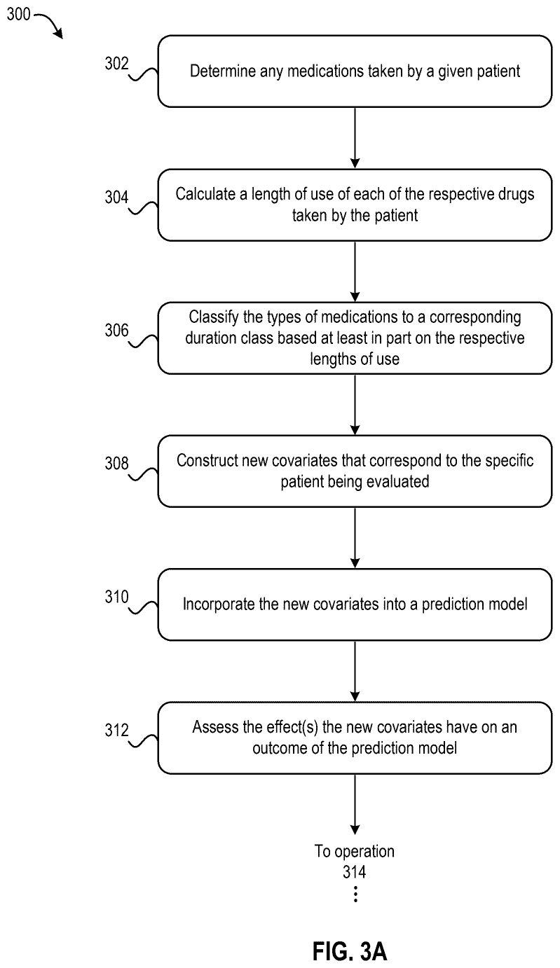

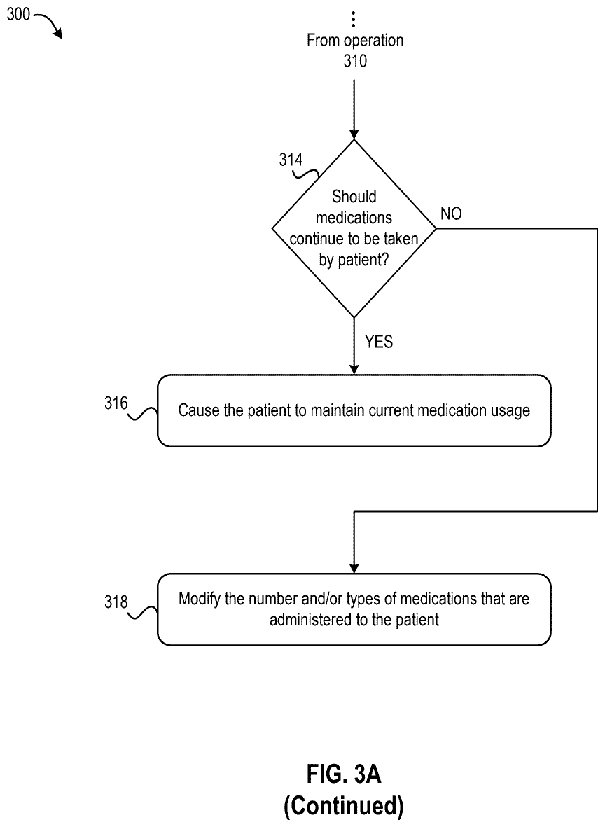

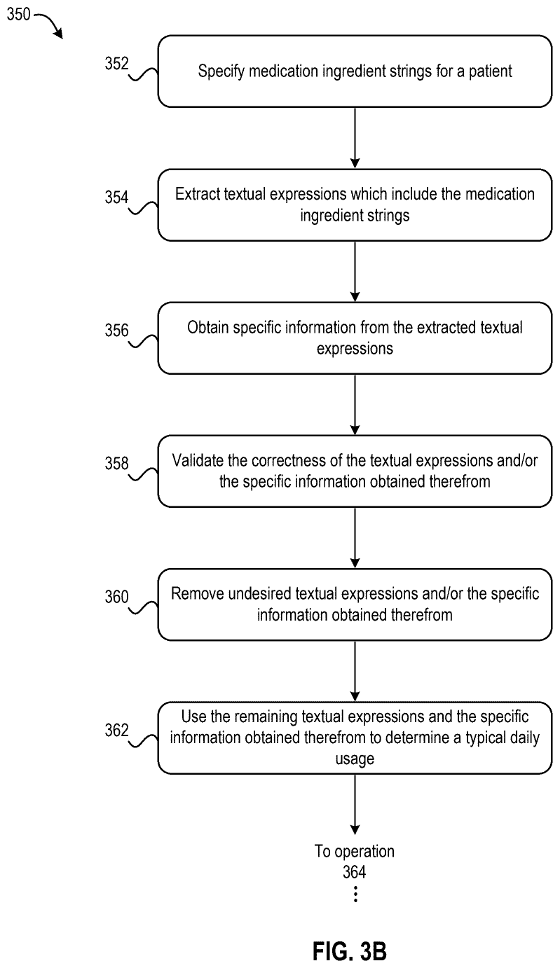

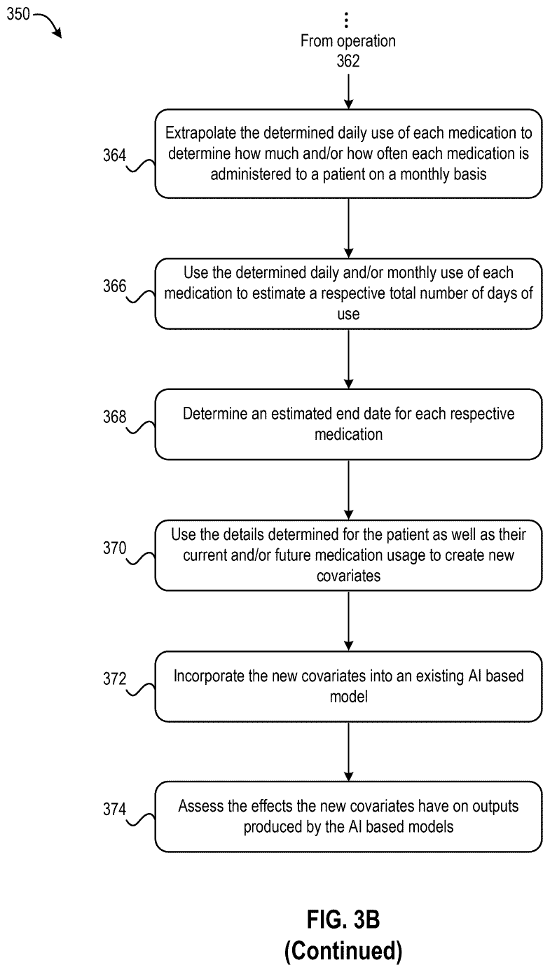

Looking now to FIG. 3A, a flowchart of a computer-implemented-method 300 for creating new covariates and integrating them into AI based models to provide more detailed insight. As noted above, the process of assessing the effect of taking a certain drug as a function of duration on patient outcomes is a complicated topic that depends on a countless number of factors and variables. Thus, the same patient being prescribed a certain drug for different durations may have drastically different impacts on the patient's health in the short and long term.

In some approaches, one or more of the operations in method 300 may be performed by an AI model that is trained using a predetermined training set of data. For example, in some approaches, various of the operations noted above may be deployed in a trained state of a trained AI model. Training of the AI model, in some approaches, may be performed by applying a predetermined training data set to develop and/or implement novel covariates which gather new insights regarding how the AI model should operate. For example, covariates corresponding to the length of time a drug configured to treat symptoms of IBD has been taken by a patient with medical issue(s) may be integrated into one or more AI based models that are able to make predictions on whether actions taken now will increase or decrease the chances of a medical outcome (such as a major surgery) in the future to treat the medical issue(s). Initial training may include reward feedback that may, in some approaches, be implemented using a subject matter expert (SME) that generally understands drugs and how they impact patients in the short and long terms.

However, to prevent costs associated with relying on manual actions of a SME, in another approach, reward feedback may be implemented using techniques for training an AI model, as would become apparent to one skilled in the art after reading the present disclosure. Once a determination is made that the AI model achieves a redeemed threshold of accuracy of performing the operations described herein during this training, a decision that the model is trained and ready to deploy for performing techniques and/or operations of method 300 may be performed. In some further approaches, the AI model may be a deep learning AI model that may improve performance of computer devices in an infrastructure associated with evaluating medical data, making future projections based at least in part on the evaluated medical data, and determining whether certain drug should be administered. The deep learning AI model may not need an SME and/or iteratively applied training with reward feedback in order to accurately perform operations described herein. Instead, the deep learning AI model is configured to itself make determinations described in operations herein.

Weight values may, in some approaches, be used by the AI reasoning model to collect and analyze information and/or feedback potentially received from a patient and/or medical professional. Such an AI model ensures that re-training occurs, during which the accuracy of the determinations generated by the AI model is evaluated. In situations where the accuracy with which the AI model is predicting future patient conditions and/or the impacts (e.g., such as re-admission, surgery, mortality, uncontrolled lab or vital observation, etc., or any other medical outcomes) that administering a given drug has on a patient, the covariates used to train the AI model may be shifted (e.g., weighted) such that the AI model(s) produce more accurate predictions and determinations as a result of the re-training, where the scale of such analysis and determinations would not otherwise be feasible for a human to perform. This is because humans are not able to efficiently weigh the impact that various medical based actions (e.g., administering a specific drug for a specific amount of time to the patient) will have on the medical condition the patient is suffering from in the short and long term. This would otherwise incorporate processing delays and errors in the process of attempting to do so. Accordingly, management of operations described herein is not able to be achieved by human manual actions.

With continued reference to FIG. 3A, the method 300 may be performed in accordance with the present invention in any of the environments depicted in FIGS. 1-2, among others, in various approaches. Of course, more or less operations than those specifically described in FIG. 3A may be included in method 300, as would be understood by one of skill in the art upon reading the present descriptions.

Each of the steps of the method 300 may be performed by any suitable component of the operating environment. For example, in some approaches one or more of the operations in method 300 may be performed by an AI based model which is implemented in an AI based module (e.g., see AI modules 213, 238 of FIG. 2). However, the method 300 may be partially or entirely performed by a controller, a processor, a computer, etc., or some other device having one or more processors therein. Moreover, the terms computer, processor and controller may be used interchangeably with regards to any of the approaches herein, such components being considered equivalents in the many various permutations of the present invention.

For those approaches having a processor, the processor, e.g., processing circuit(s), chip(s), and/or module(s) implemented in hardware and/or software, and preferably having at least one hardware component may be utilized in any device to perform one or more steps of the method 300. Illustrative processors include, but are not limited to, a central processing unit (CPU), an application specific integrated circuit (ASIC), a field programmable gate array (FPGA), etc., combinations thereof, or any other suitable computing device known in the art.

As shown, operation 302 includes determining any drugs taken (e.g., administered to) by a given patient. For instance, operation 302 includes identifying any ingredients, brand names, common side-effects, manufacturing date, etc. of any drugs that are currently being taken by the patient. In other words, operation 302 includes collecting any relevant information pertaining to the drugs that an individual is taken (or has taken). Furthermore, operation 304 includes calculating a length of use (e.g., a number of days) of each of the respective drugs taken by the patient (considering also dose per tablet). While drugs are used to treat medical conditions, use of these drugs may have unintended effects. For example, prolonged use of a specific prescription drug configured to treat symptoms of a medical issue (e.g., IBD) may ultimately cause a patient's health to suffer in the long term. Knowing how long a patient has taken a drug or intends to take a drug may thereby be evaluated in order to avoid serious issues for the patient in the future. Incorporating this understanding of length of use allows AI based models to make determinations on how a patient should be treated to reduce the risk or even avoid future medical issues, e.g., which may call for major surgery to be conducted.

In some approaches, operation 302 and/or operation 304 may include extracting prescription dates and/or quantities for each of the respective drugs (configured to treat symptoms of one or more medical issues) taken by the patient. In other words, operation 302 and/or operation 304 involves determining a quantifiable amount of each respective drug the patient is administered (e.g., given) over a given period. The prescription dates and/or quantities may be extracted from the patient's medical records, publicly available information (e.g., recommended dosages for a specific drug), the patient themselves (e.g., using a questionnaire), by evaluating samples (e.g., blood, saliva, etc.) taken from the patient, etc.

These details gleaned in operations 302 and 304 may thereby be evaluated to generate a summary (e.g., approximation) of what drugs the patient takes on a daily, weekly, bi-weekly, monthly, annual, semi-annual, etc. basis. For instance, the respective quantities of the drugs may be evaluated along with the determined typical daily use to calculate estimated end dates for the respective drugs (as end date data entries may not be available in the medical databases). Moreover, the end dates may be used to estimate (e.g., determine) a length of use for each drug. In other words, operations 302 and/or 304 may be able to determine how much of each drug will be received (e.g., ingested) by the patient over a predetermined period (e.g., their life). As noted above, the length of time a patient takes one or more drugs may have an impact on their overall health and/or the status of conditions that caused the drugs to be prescribed in the first place, e.g., to treat symptoms of one or more medical issues.

From operation 304, method 300 advances to operation 306. There, operation 306 includes classifying the types of drugs to a corresponding duration class based at least in part on the respective lengths of use. In other words, operation 306 includes grouping the different drugs taken by the patient based at least in part on the type of drugs and the respective lengths of time that the patient has been taking the different types of drugs. In some approaches, the drugs may be grouped into different duration classes that are formed for the specific patient based on an age, gender, location, habits, etc. of a patient. In other approaches, the duration classes may be obtained from publicly available information. In other words, the duration classes may correspond to ranges that are predetermined based on industry standards. For example, the duration classes may be obtained (e.g., extracted) at least in part from information included in one or more disease registries.

From operation 306, method 300 advances to operation 308. There, operation 308 includes constructing new covariates that correspond to the specific patient being evaluated. In other words, operation 308 includes identifying details that pertain to a specific patient and the drugs they are taking. The new covariates may be developed based at least in part on the types of drugs currently and/or previously taken by the patient as well as the respective lengths of use for the types of drugs. These covariates thereby provide insight into what external factors are impacting the health of the patient.

From operation 308, method 300 advances to operation 310. There, operation 310 includes incorporating the new covariates into a prediction model. In other words, operation 310 includes training (or re-training) one or more AI based models such that they consider the new covariates (e.g., along with standard covariates already incorporated into the AI based model(s)) during the process of generating a determination. For example, AI based models may be trained using the new covariates along with other existing covariates to determine if and/or how long a patient should be administered one or more specific drugs (e.g., prescription drugs).

Incorporating these new covariates into one or more AI based models desirably improves the accuracy with which the models are able to generate predictions of how current actions will impact future health of patients. As noted above, the integration of new and useful covariates into a prediction model (AI based model) improves the accuracy and detail of the model. For example, novel time dependent covariates (e.g., such as indicating different lengths of use per drug type) may be developed in approaches herein and used to gather new insights regarding the impact that determinations made by one or more AI based models have on the patient in the short and/or long term.

Accordingly, operation 312 includes assessing the effect(s) the new covariates have on an outcome of the prediction model. Evaluating the effect(s) of the new covariates provides valuable insight as to how accurately the model is able to predict the impact different medical based actions (e.g., administering one or more prescription drugs configured to treat symptoms of one or more medical issues) have on a patient. Approaches herein are thereby able to improve the accuracy with which determinations are made on how a patient should be treated for an underlying medical condition.

For instance, operation 312 may include using the new covariates to identify specific ones of the drugs (e.g., drugs) taken by the patient, that are of interest. In other words, the new covariates are used to form new subpopulations of drugs in the set of drugs. These specific drugs may be grouped into respective subpopulations that are evaluated independently. Different drugs have different effects on a patient. For example, the side effects of some drugs make them incompatible with others. Thus, by combining different groupings of drugs, the AI based models herein are able to evaluate details that are specific to each group of drugs. According to a non-limiting example, subpopulations of drugs may be grouped based on the respective active ingredient(s) therein. Evaluating the groups of drugs independently (and/or in combination) may thereby allow the AI based models to determine whether continued use of the drugs will have a negative impact on the patient's overall health and/or specific condition being treated, e.g., as would be appreciated by one skilled in the art after reading the present description.

In some approaches, operation 312 may include evaluating training data using the AI based model having the new covariates. This may provide an opportunity to test the accuracy with which the model is able to interpret information available for a patient and make predictions on how current actions will impact the effects a medical condition has on the patient in the long term. Determinations made by the AI based model may further be compared against known outcomes corresponding to the training data, e.g., to compute the accuracy of the model.

Referring still to FIG. 3A, method 300 advances from operation 312 to operation 314. There, operation 314 includes using the AI based models to determine whether a prescription should be extended for a patient, e.g., such that the patient may continue to take the prescribed drug(s). In other words, operations 312 and 314 includes using the AI based models having the newly incorporated covariates to determine an amount of time that the patient should use each of their respective drugs. In some approaches, the determination made in operation 314 may be based at least in part on whether the new covariates incorporated into the AI based models in operation 310 have an effect on the prediction generated by the AI based models. In other words, operation 314 may consider whether the new covariates cause the output produced by the AI based models to change for the better or worse.

In response to determining that incorporating the new covariates has not impacted outputs produced by the AI based models, method 300 advances from operation 314 to operation 316. There, operation 316 includes causing at least one of the drugs in the determined set of drugs to continue being administered to the patient. In other words, operation 316 includes causing the patient to maintain current drug usage of at least one of the drugs identified in the set in operation 302. In some approaches, operation 316 includes causing the patient to maintain current drug usage. In other words, operation 316 includes continuing to administer each of the drugs identified in operation 302 to the patient. Operation 316 may thereby include following prescriptions previously provided by a medical professional. In other approaches, the prescription for one or more of the drugs in the set identified in operation 302 may be revoked, thereby preventing the patient from continued use thereof.

However, in response to determining that incorporating the new covariates has impacted outputs produced by the AI based models, method 300 advances from operation 314 to operation 318. There, operation 318 includes modifying the number and/or types of drugs that are administered to the patient. Operation 318 may further include modifying the amounts of the drugs that are administered, the frequency at which the drugs are administered, the order in which the drugs are administered, etc. In some approaches, operation 318 includes sending one or more instructions to a medical professional (e.g., doctor), making modifications to the patient's medical records, submitting and/or modifying prescriptions that are sent to a pharmacist for fulfillment, scheduling an appointment for the patient such that prescribed drugs may be administered by a medical professional, etc.

Again, method 300 is desirably able to develop AI based models that have been trained to consider the different types of drugs used by (e.g., administered to) a patient, as well as the respective lengths that the drugs have been used by the patient. It follows that approaches herein are able to create new covariates and integrate them into AI based models to provide more detailed insight. These new covariates gather new insights regarding the impact they have on determinations that are made by the one or more models. For example, covariates corresponding to the length of time a drug (e.g., prescription drug) have been taken by a patient with medical issue(s) may be integrated into one or more AI based models that are able to make predictions on whether actions taken now will increase or decrease the chances of a major surgery in the future to treat the medical issue(s). Approaches herein may thereby be modified to improve the process of prescribing medical treatment (e.g., medical prescriptions) and/or determining whether to cause the medical treatment to be administered to a patient.

According to an example, which is in no way intended to be limiting, the AI based models are able to evaluate details corresponding to using different drugs for different lengths of time, and make determinations (e.g., informed predictions) that outline how using each of the drugs for different lengths of time will impact the long term health of the patient, particularly with respect to an underlying issue (e.g., medical condition) being treated. For instance, the AI based models may be able to determine that taking a prescription drug for a short period of time will actually have a more desirable (e.g., positive) impact on a patient's condition than prolonged use of the prescribed drug.

Looking now to FIG. 3B, a method 350 for creating new covariates and integrating them into AI based models to provide more detailed insight is illustrated in accordance with another approach. Again, the process of assessing the effect of taking a certain drug as a function of duration on patient outcomes is a complicated topic that depends on a countless number of factors and variables.

The method 350 may be performed in accordance with the present invention in any of the environments depicted in FIGS. 1-2, among others, in various approaches. Of course, more or less operations than those specifically described in FIG. 3B may be included in method 350, as would be understood by one of skill in the art upon reading the present descriptions.

Each of the steps of the method 350 may be performed by any suitable component of the operating environment. For example, in some approaches one or more of the operations in method 350 may be performed by a AI based model which is implemented in an AI based module (e.g., see AI modules 213, 238 of FIG. 2). However, the method 350 may be partially or entirely performed by a controller, a processor, a computer, etc., or some other device having one or more processors therein. Moreover, the terms computer, processor and controller may be used interchangeably with regards to any of the approaches herein, such components being considered equivalents in the many various permutations of the present invention.

For those approaches having a processor, the processor, e.g., processing circuit(s), chip(s), and/or module(s) implemented in hardware and/or software, and preferably having at least one hardware component may be utilized in any device to perform one or more steps of the method 350. Illustrative processors include, but are not limited to, a central processing unit (CPU), an application specific integrated circuit (ASIC), a field programmable gate array (FPGA), etc., combinations thereof, or any other suitable computing device known in the art.

As shown, operation 352 includes specifying drug ingredient strings for a patient. In other words, operation 352 includes identifying different drugs that a given patient is taking. The drugs may be identified using a brand name, active ingredient(s) therein, generic drug names, etc. Moreover, the different drugs in the ingredient strings may be identified from medical charts, notes from a physician, forms (e.g., intake forms) filled out by the patient, etc.

Operation 354 further includes extracting textual expressions which include the drug ingredient strings, as well as other data elements such as dose and unit of the dose. In other words, operation 354 includes identifying and extracting any alphanumeric characters from a source that includes the drug ingredient strings. Operation 356 further includes obtaining specific information from the extracted textual expressions. In some approaches, operation 356 includes using the extracted textual expressions to determine the specific doses of the respective drugs that are administered to the patient. In some approaches, operation 356 includes using the extracted textual expressions to determine the specific quantities and/or frequencies that the respective drugs are administered to the patient. The specific information may be obtained from the extracted textual expressions using text processing.

Advancing to operation 358, method 350 includes validating the correctness of the textual expressions and/or the specific information obtained therefrom. In other words, operation 358 includes comparing the textual expressions and/or specific information to previously predicted values, medical information of other patients with the same condition(s) and/or taking the same drug(s), industry based medical standards, user provided inputs on how they are feeling, etc. Furthermore, operation 360 includes removing undesired textual expressions and/or the specific information obtained therefrom (such as “drops”, “injection”, “cream”, “powder”). Operation 360 may thereby include removing textual expressions and/or the specific information determined to be irrelevant or incorrect for the given patient. Operation 360 may be performed by comparing the textual expressions and/or the specific information obtained therefrom to medical records, previous iterations of method 300 and/or method 350, inputs provided by the patient, etc.

From operation 360, method 350 proceeds to operation 362. There, operation 362 includes using the remaining textual expressions and the specific information obtained therefrom to determine a typical daily usage. In other words, operation 362 includes evaluating the extracted information to determine how much and/or how often each drug is administered to a patient on a daily basis (which may be normalized according to dose). Operation 364 further includes extrapolating this determined daily use of each drug to determine how much and/or how often each drug is administered to a patient on a monthly basis.

The determined daily and/or monthly use of each drug are further used (e.g., in combination with the determined quantities of the respective drugs administered to the patient) to estimate lengths of use of the respective drugs. In other words, the daily and/or monthly use are used to determine a respective total number of days of use for each of the respective drugs. See operation 366. The information pertaining to use of the drugs determined in operations 362 and/or 364 may thereby be used to determine a total number of days the patient will receive (e.g., ingest, absorb, etc.) the drugs. Furthermore, operation 368 includes using the estimated lengths of use and the prescription dates to determine respective prescription end dates. In other words, operation 368 includes determining an estimated end date for each respective drug. Operation 368 thereby utilizes the prescription issue date, estimated number of days of use, etc. to identify a future date that the drug will no longer be administered to the patient.

As noted above, the total number of days a patient takes a drug has an impact on how the patient reacts in the short and long term. Accordingly, operation 370 further includes using the details determined for the patient as well as their current and/or future drug usage to create new covariates. These new covariates may be created by comparing and/or combining different drug based details for the given patient. Moreover, operation 372 includes incorporating the new covariates into an existing AI based model, while operation 374 includes assessing the effects the new covariates have on outputs produced by the AI based models. For example, operation 374 may include determining and/or applying odds ratios to the determined drug usage details.

Again, approaches herein are desirably able to generate a new types of covariates. Moreover, incorporating the new covariates into AI based models (e.g., a prediction model) allows for approaches herein to measure the impact of taking a certain drug for a given duration of time. Approaches herein are thereby able to quantitatively measure the magnitude of the effect that length-of-use has on the patient.

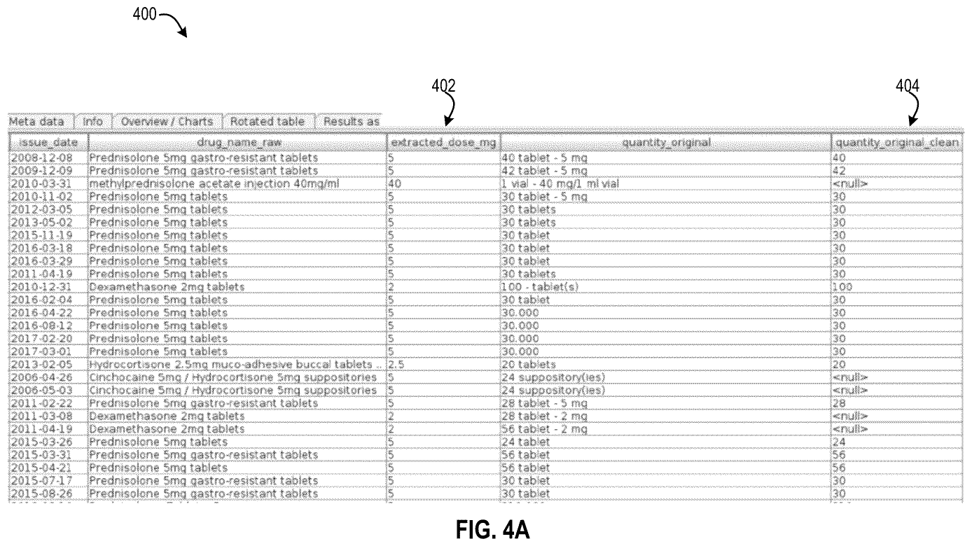

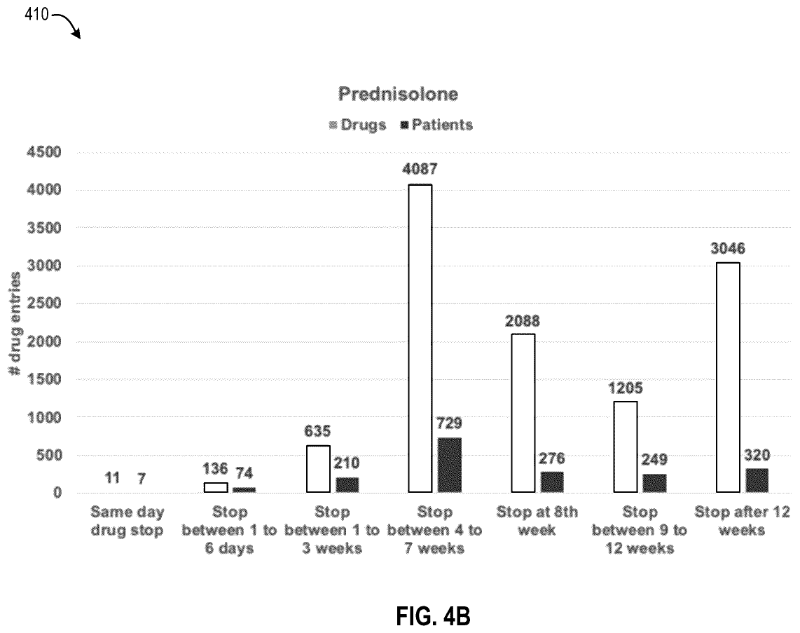

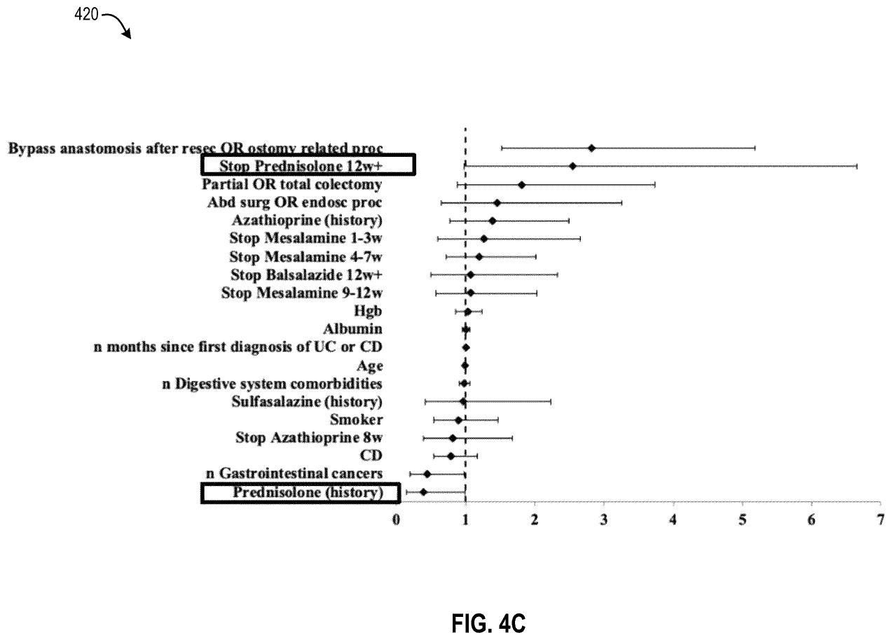

Looking now to FIGS. 4A-4C, results output by one or more of the AI based models herein in response to evaluating details pertaining to a given patient suffering from a GI based medical issue are illustrated in accordance with an in-use example, which is in no way intended to be limiting.

Looking first to FIG. 4A, table 400 illustrates how details corresponding to a patient may be evaluated to determine the dosage and/or quantity of a drug being administered to the patient. Specifically, table 400 includes prescription issue dates along with respective prescription drug names. As shown, this information may be evaluated and used to determine the actual dosage 402 and quantity 404 of the respective drugs. This may be achieved using any of the approaches herein.

Looking now to FIG. 4B, graph 410 illustrates exemplary 7 duration classes for use of PREDNISOLONE among various patients (i.e., resulting 7 new covariates). While several specific duration classes are shown in graph 410, these are in no way intended to be limiting. Rather, the duration classes may vary depending on the patient, the drug, a prescribing physician, the type of AI based model(s) being used, etc.

Furthermore, FIG. 4C depicts another graph 420 which shows the impact that taking different prescription drugs for different lengths of time will have on a patient. Specifically, graph 420 illustrates how taking certain prescription drugs for different amounts of time and/or specific procedures increase or decrease the likelihood of a patient being prescribed a major surgery in the future. Graph 420 may be produced by one or more of the AI based models herein, e.g., in response to performing method 300 and/or method 350.

Graph 420 shows that performing a bypass with anastomosis after resection has a similar effect as an ostomy related procedure. Specifically, these available options strongly increase the odds that the patient will undergo a major surgery in the future. Graph 420 also illustrates that use of PREDNISOLONE generally reduces the odds that the patient will undergo a major surgery in the future. However, graph 420 shows that prolonged use of PREDNISOLONE actually increases the odds that the patient will undergo a major surgery in the future.

It will be clear that the various features of the foregoing systems and/or methodologies may be combined in any way, creating a plurality of combinations from the descriptions presented above.

It will be further appreciated that implementations of the present invention may be provided in the form of a service deployed on behalf of a customer to offer service on demand.

The descriptions of the various implementations of the present invention have been presented for purposes of illustration, but are not intended to be exhaustive or limited to the implementations disclosed. Many modifications and variations will be apparent to those of ordinary skill in the art without departing from the scope and spirit of the described implementations. The terminology used herein was chosen to best explain the principles of the implementations, the practical application or technical improvement over technologies found in the marketplace, or to enable others of ordinary skill in the art to understand the implementations disclosed herein.

What is claimed is:

1. A method comprising:
defining ingredients and brand names of the ingredients in a set of drugs;
calculating lengths of use of the respective drugs in the set;
classifying types of the drugs, and the respective lengths of use, to a corresponding duration class;
constructing new covariates based at least in part on the types of drugs and/or the respective lengths of use;
incorporating the new covariates and standard covariates into a prediction model, wherein the prediction model is an artificial intelligence (AI) based model created and maintained by using a predetermined training set of data to train the AI based model to consider: types of drugs used, and respective lengths of use, in determining whether a prescription should be extended for a patient; and
assessing effect(s) the new covariates have on an outcome of the AI based model.

2. The method of claim 1, further comprising, for the drugs in the set of drugs:
extracting respective prescription dates;
extracting respective quantities;
generating typical daily use of the respective drugs;
combining the respective quantities and the typical daily use to calculate estimated lengths of use of the respective drugs;
using the estimated lengths of use and the prescription dates to determine respective prescription end dates; and
using the prescription end dates to calculate the respective lengths of use.

3. The method of claim 1, further comprising:
using the new covariates to form new subpopulations of drugs in the set of drugs;
causing the new subpopulations of drugs to be analyzed independently; and
re-training the trained AI based model using feedback received from a patient and/or medical professional regarding the new covariates and new subpopulations of drugs.

4. The method of claim 1, wherein the new covariates are constructed based at least in part on the types of drugs and the respective lengths of use.

5. The method of claim 1, wherein the classifying the types of the drugs, and the respective lengths of use, to a corresponding duration class includes:
extracting duration classes from one or more disease registries.

6. The method of claim 1, further comprising:
using feedback received from a patient and/or medical professional regarding the new covariates and new subpopulations of drugs to re-train the trained AI based model; and
evaluating accuracy of determinations generated by the re-trained AI model.

7. The method of claim 6, wherein the prescription is for a drug configured to treat symptoms of inflammatory bowel disease.

8. The method of claim 7, further comprising:
in response to determining the new covariates do not have an effect on the outcome of the AI based model, causing the prescription to be extended for the patient, and causing a drug corresponding to the prescription to be administered to the patient.

9. The method of claim 1, further comprising:
in response to determining the new covariates do not have an impact on the outcome of the AI based model, causing at least one drug in the set of drugs to continue to be administered to a patient.

10. A computer program product comprising:
one or more computer-readable storage media; and
program instructions stored on the one or more storage media to perform operations comprising:
defining ingredients and brand names of the ingredients in a set of drugs;
calculating lengths of use of the respective drugs in the set;
classifying types of the drugs, and the respective lengths of use, to a corresponding duration class;
constructing new covariates based at least in part on the types of drugs and/or the respective lengths of use;
incorporating the new covariates and standard covariates into a prediction model, wherein the prediction model is an artificial intelligence (AI) based model created and maintained by using a predetermined training set of data to train the AI based model to consider: types of drugs used, and respective lengths of use, in determining whether a prescription should be extended for a patient; and
assessing effect(s) the new covariates have on an outcome of the AI based model.

11. The computer program product of claim 10, wherein the operations further comprise, for the drugs in the set of drugs:
extracting respective prescription dates;
extracting respective quantities;
generating typical daily use of the respective drugs;
combining the respective quantities and the typical daily use to calculate estimated lengths of use of the respective drugs;
using the estimated lengths of use and the prescription dates to determine respective prescription end dates; and
using the prescription end dates to calculate the respective lengths of use.

12. The computer program product of claim 10,
wherein the operations further comprise:
using the new covariates to form new subpopulations of drugs in the set of drugs;
causing the new subpopulations of drugs to be analyzed independently; and
re-training the trained AI based model using feedback received from a patient and/or medical professional regarding the new covariates and new subpopulations of drugs.

13. The computer program product of claim 10, wherein the new covariates are constructed based at least in part on the types of drugs and the respective lengths of use.

14. The computer program product of claim 10, wherein the classifying the types of the drugs, and the respective lengths of use, to a corresponding duration class includes:
extracting duration classes from one or more disease registries.

15. The computer program product of claim 10, wherein the operations further comprise:
using feedback received from a patient and/or medical professional regarding the new covariates and new subpopulations of drugs to re-train the trained AI based model; and
evaluating accuracy of determinations generated by the re-trained AI model.

16. The computer program product of claim 15, wherein the prescription is for a drug configured to treat symptoms of inflammatory bowel disease.

17. The computer program product of claim 16, wherein the operations further comprise:
in response to determining the new covariates do not have an effect on the outcome of the AI based model, causing the prescription to be extended for the patient, and causing a drug corresponding to the prescription to be administered to the patient.

18. The computer program product of claim 10,
wherein the operations further comprise:
in response to determining the new covariates do not have an impact on the outcome of the AI based model, causing at least one drug in the set of drugs to continue to be administered to a patient.

19. A computer system comprising:
a processor set;
one or more computer-readable storage media; and
program instructions stored on the one or more storage media to cause the processor set to perform operations comprising:
defining ingredients and brand names of the ingredients in a set of drugs;
calculating lengths of use of the respective drugs in the set;
classifying types of the drugs, and the respective lengths of use, to a corresponding duration class;
constructing new covariates based at least in part on the types of drugs and/or the respective lengths of use;
incorporating the new covariates and standard covariates into a prediction model, wherein the prediction model is an artificial intelligence (AI) based model created and maintained by using a predetermined training set of data to train the AI based model to consider: types of drugs used, and respective lengths of use, in determining whether a prescription should be extended for a patient; and
assessing effect(s) the new covariates have on an outcome of the AI based model.

20. The computer system of claim 19, wherein the operations further comprise:
in response to determining the new covariates do not have an impact on the outcome of the AI based model, causing at least one drug in the set of drugs to continue to be administered to a patient.
